(19) United States
(12) Patent Application Publication
(10) Pub. No.: US 2008/0027272 A1
(43) Pub. Date: Jan. 31, 2008

(54) Device for processing blood vessel harvested for bypass graft surgery

(75) Inventor: Randal James Kadykowski, South Lyon, MI (US)

Correspondence Address: TERUMO CARDIOVASCULAR SYSTEMS CORPORATION;ATTN: GAEL DIANE TISACK, 6200 JACKSON ROAD, ANN ARBOR, MI, 48103, US

(73) Assignee: Terumo Cardiovascular Systems Corporation

(21) Appl. No.: 11/493,319

(22) Filed: Jul. 26, 2006

(51) Int. Cl. A61F 2/04

(52) U.S. Cl. 600/36

(57) ABSTRACT

A vessel holder supports a blood vessel during processing for use in bypass graft surgery. The holder comprises a base and a first fixture mounted on the base for holding a first end of the blood vessel. The first fixture has a fluid supply needle for inserting into the first end of the blood vessel. A second fixture is mounted on the base a predetermined distance from the first fixture for holding a second end of the blood vessel. The predetermined distance is adjustable for holding the blood vessel under tension. The device reduces labor and time for vessel preparation. With various optional features, the device obtains safer application of pressurized saline solution when testing for leaks, better visualization for inspecting the vessel for surgical use, reliable protection of the vessel from accidental damage, and convenient immersion of the vessel in a bath for preservation until needed in the surgery.

### CROSS REFERENCE TO RELATED APPLICATIONS

Not Applicable.

### STATEMENT REGARDING FEDERALLY SPONSORED RESEARCH

Not Applicable.

### BACKGROUND OF THE INVENTION

The present invention relates in general to a holder for a blood vessel harvested from a patient for use in cardiac bypass graft surgery, and, more specifically, to a device for assisting in sealing branch stubs and pressure testing of the blood vessel.

In coronary artery bypass grafting (CABG), a blood vessel or vessel section, such as an artery or vein, is “harvested” (i.e., removed) from its natural location in a patient's body for use elsewhere in the body. In CABG surgery, the blood vessel is used to form a bypass between an arterial blood source and the coronary artery that is to be bypassed. Among the preferred sources for the vessel to be used as the bypass graft are the saphenous veins in the legs and the radial artery in the arms.

Endoscopic surgical procedures for harvesting a section of a vein (e.g., the saphenous vein) subcutaneously have been developed in order to avoid disadvantages and potential complications of harvesting through a continuous incision (e.g., along the leg) for the full length of the desired vein section in order to provide adequate exposure for visualizing the vein and for introducing surgical instruments to sever, cauterize and ligate the tissue and side branches of the vein. One such minimally-invasive technique employs a small incision for locating the desired vein and for introducing one or more endoscopic harvesting devices. Primary dissection occurs by introduction of a dissecting instrument through the incision to create a working space and separate the vein from the surrounding tissue. Then a cutting instrument is introduced into the working space to sever the blood vessel from the connective tissue surrounding the section to be harvested and any side branches of the blood vessel. The branches may be clipped and/or cauterized.

In one typical procedure, the endoscopic entry site is located near the midpoint of the vessel being harvested, with dissection and cutting of branches proceeding in both directions along the vessel from the entry site. In order to remove the desired section of the blood vessel, a second small incision, or stab wound, is made at one end thereof and the blood vessel section is ligated. A third small incision is made at the other end of the blood vessel section which is then ligated, thereby allowing the desired section to be completely removed through the first incision. Alternatively, only the first two incisions may be necessary if the length of the endoscopic device is sufficient to obtain the desired length of the blood vessel while working in only one direction along the vessel from the entry point.

An example of a commercially available product for performing the endoscopic vein harvesting described above is the VirtuoSaph™ Endoscopic Vein Harvesting System from Terumo Cardiovascular Systems Corporation of Ann Arbor, Mich. Endoscopic vein harvesting systems are also shown in U.S. Pat. No. 6,660,016 to Lindsay and U.S. patent application publication 2005/0159764A1 in the name of Kasahara et al, both of which are incorporated herein by reference in their entirety.

After a vein is removed from the patient's body, it must be prepared for use as a bypass graft. Preparation includes ligating (i.e., closing off) each branch stub, injecting a solution into the vein under pressure to test for leaks, and otherwise inspecting the condition of the blood vessel. In the conventional procedures, branch stubs may be ligated using sutures for tying off each stub or staples may be employed for clamping off each stub. This processing of the blood vessel is normally performed by placing the blood vessel on a sterile surface over a patient's leg nearby the point from where it was harvested. Typically, two people (e.g., medical technicians, nurses) work together to hold the vein, apply a syringe of saline solution to pressurize the vessel, tie off or clip branch stubs as the vein is distended by the saline solution, and check for leaks. The prior art procedure is labor intensive and is difficult to perform on a loose, unsupported vein lying over an uneven surface. Since the amount of pressurized saline solution injected into a blood vessel is manually controlled according to the pressure being applied against a plunger of a syringe, the medical technician occasionally damages a blood vessel by over-pressurizing it. Other inadvertent damage may also occur during handling since the blood vessel is unrestrained and unprotected from other objects until such time as it is transferred to a bath of saline solution after it has been inspected by a surgeon performing the graft.

### SUMMARY OF THE INVENTION

Among the benefits that can be realized according to different aspects of the invention are reduced labor and time for vessel preparation, safer application of pressurized saline solution when testing for leaks, better visualization for inspecting and measuring the vessel for surgical use, reliable protection of the vessel from accidental damage, and convenient immersion of the vessel in a bath for preservation until needed in the surgery.

In one aspect of the invention, a vessel holder supports a blood vessel during processing for use in bypass graft surgery. The holder comprises a base and a first fixture mounted on the base for holding a first end of the blood vessel. The first fixture has a fluid supply needle for inserting into the first end of the blood vessel. A second fixture is mounted on the base a predetermined distance from the first fixture for holding a second end of the blood vessel. The predetermined distance is adjustable for holding the blood vessel under tension.

### BRIEF DESCRIPTION OF THE DRAWINGS

FIG. 1 shows a saphenous vein being removed from the leg of a patient following an endoscopic procedure.

FIG. 2 is a perspective view of one embodiment of a vessel holder according to the present invention.

FIG. 3 is a perspective view of another embodiment of the vessel holder having an intermediate branch clamp.

FIG. 4 is a perspective view of a further embodiment of the invention having a removable cover that is used to provide a bath.

FIG. 5 is a cross-sectional perspective view according to another embodiment of the present invention.

FIG. 6 is a perspective view of another embodiment of the vessel holder.

FIG. 7 shows a modification to the embodiment of FIG. 6 including a vessel bath with an adjustable length.

FIG. 8 is a perspective view of a further embodiment of the invention having a sleeve for fitting over a limb of the patient.

FIG. 9 is a perspective view of yet another embodiment of the vessel holder having graduation marks and labels for assisting in determining the length of a vessel being prepared.

FIG. 10 is a partial view of the vessel holder in FIG. 9 wherein a reservoir holds a prepared blood vessel in a bath of saline solution until needed for the surgery.

### DETAILED DESCRIPTION OF PREFERRED EMBODIMENTS

The present invention provides a device for supporting a blood vessel during processing for use in bypass graft surgery. The vessel holder device comprises a base and first and second fixtures mounted on the base. The first fixture holds a first end of the blood vessel and has a fluid supply needle for inserting into the first end of the blood vessel. The second fixture holds a second end of the blood vessel at a predetermined distance from the first fixture. The predetermined distance between the first and second fixtures is adjustable so that the blood vessel is held under a desired amount of tension. By suspending the blood vessel with a gentle tension between the first and second fixtures, processing of a blood vessel can be performed by a single individual in a shorter period of time and with less damage to the blood vessel.

Referring now to FIG. 1, a saphenous vein 10 is being removed from a patient's leg 11 through an incision 12. During harvesting, main vessel 10 is severed from side branches extending from vessel 10 and then opposite ends of vessel 10 are cut at stab wounds 13 and 14 to free it for removal. The present invention may also be employed with blood vessels harvested using different surgical methods or from different areas of the patient's body.

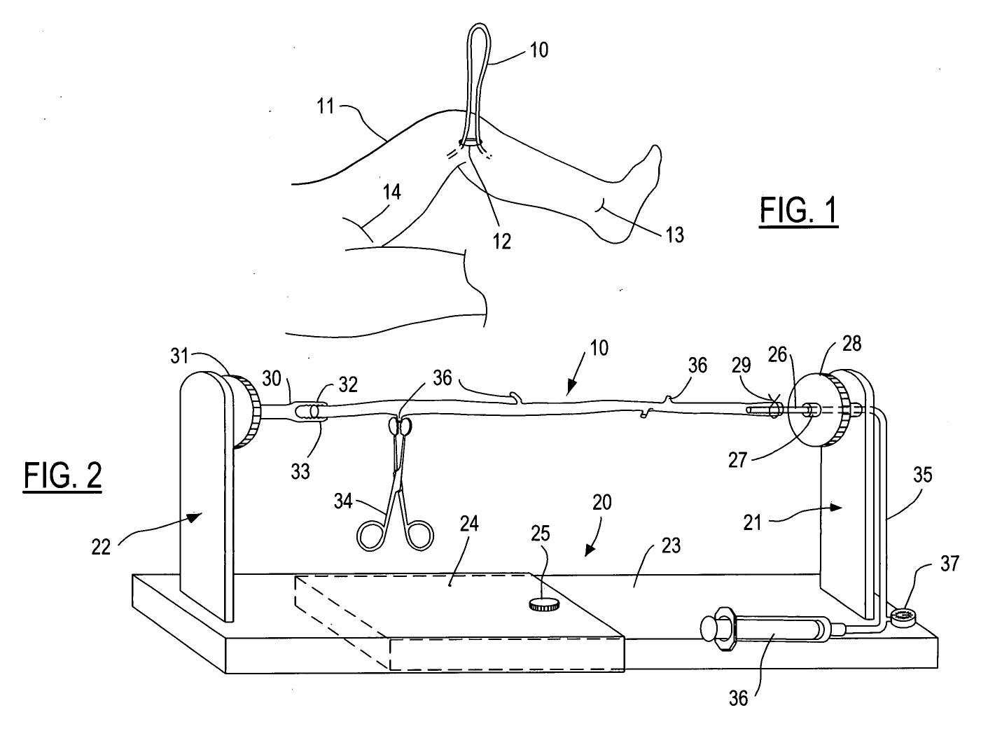

The present invention may include a vessel holder device supplied as a disposable, sterile device for use by a physician's assistant or nurse to prepare a blood vessel. In a preferred embodiment, the device is constructed as a disposable stand molded from polycarbonate or other biocompatible material and using stainless steel attachment devices arranged to apply the necessary tension to the blood vessel to keep it taut in order to make tying or clipping of branch stubs easier than in the prior art wherein a loose blood vessel lies on an uneven leg surface. According to a first embodiment, the device in FIG. 2 includes a base 20 supporting a first fixture 21 and a second fixture 22 wherein base 20 includes interlocking pieces 23 and 24 to adjust the distance between fixtures 21 and 22. A locking mechanism of any known type such as a locking tab 25 is provided to maintain the desired predetermined distance according to the tension to be applied.

First fixture 21 includes a fluid supply needle 26 extending from a mounting boss 27 rotationally mounted to fixture 21. A rotation wheel 28 fixed to boss 27 allows for manual rotation of fluid needle 26 and boss 27. One end of blood vessel 10 is inserted over fluid supply needle 26 and is secured thereto by tying a suture 29 over the end of blood vessel 10 and tightening.

Second fixture 22 has a clamp 30 rotationally mounted thereon together with a rotation wheel 31. Clamp 30 has a pair of jaws 32 and 33 for grasping the end of blood vessel 10. With the first end of blood vessel 10 secured to needle 26, the distance between fixtures is adjusted (if need be) to a distance at which there is sufficient slack in vessel 10 to secure it to clamp 30. After attaching both ends of blood vessel 10, the length of base 20 is adjusted outward in order to provide a predetermined distance between fixtures 21 and 22 so that blood vessel 10 is held under a desired, gentle tension. With blood vessel 10 suspended between fixtures 21 and 22, each branch stub can be conveniently tied off or clipped by a single user. Blood vessel 10 can easily be reoriented by rotating it so that a desired branch stub is facing the user by moving rotation wheels 28 and 31 together. An additional manual clamp 34 may be attached to a branch stub during ligation to pull it perpendicularly away from the main vessel in order to make suturing easier.

Boss 27 and fluid supply needle 26 include a hollow passage coupled to a feed line 35 running from a syringe 36 mounted to base 20. Thus, saline solution from syringe 36 can be coupled through feed line 35, boss 27, and needle 26 to the interior of blood vessel 10. Saline solution or other fluid may be introduced into blood vessel 10 to gradually distend it along its length to each branch stub 36 for easier ligating of the branch stubs. In addition, fluid from feed line 35 is introduced into blood vessel 10 after completing all ligations in order to test them for leaks. In order to ensure that excessive pressures that may otherwise damage blood vessel 10 are not inadvertently introduced, a pressure gauge 37 is connected to feed line 35 allowing the user to monitor pressure being applied to the vessel and to maintain it at a safe level below a known threshold.

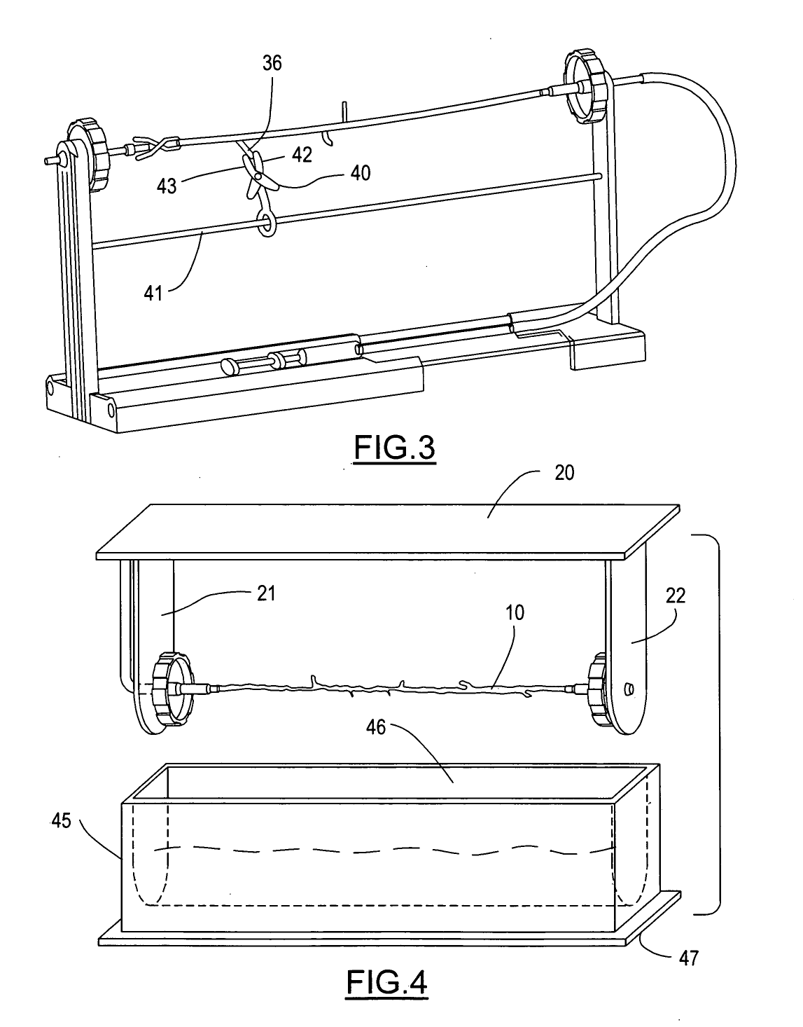

FIG. 3 shows an alternative embodiment having an additional feature of providing a branch clamp 40 that is movable along a support rod 41. Clamp 40 includes jaws 42 and 43 for grasping a branch stub 36 so that branch stub 36 is positively maintained in place to facilitate the tying of a suture around the stub. To process a next branch stub, the user removes branch clip 40 from one branch stub, rotates the rotation wheels to orient a next branch stub in the direction towards support rod 41, and clamp 40 is slid along support rod 41 so that it can easily grasp the next branch stub.

FIG. 4 shows an optional enhancement to the present invention for maintaining blood vessel 10 in a saline solution bath after the ligation and pressure testing steps. Thus, the vessel holder device may be flipped over and placed into a cover 45 having a reservoir 46 containing a sufficient depth of saline solution to immerse blood vessel 10 when the vessel holder is flipped over and inserted into reservoir 46. A flat block 47 may be provided at the bottom of cover 45 to maintain reservoir 46 in the upright position. Cover 45 may preferably be comprised of an injection molded polycarbonate or other biocompatible materials. Cover 45 and base 20 may fit together in a manner that preserves sterility during shipment from the point of manufacture to the point of use.

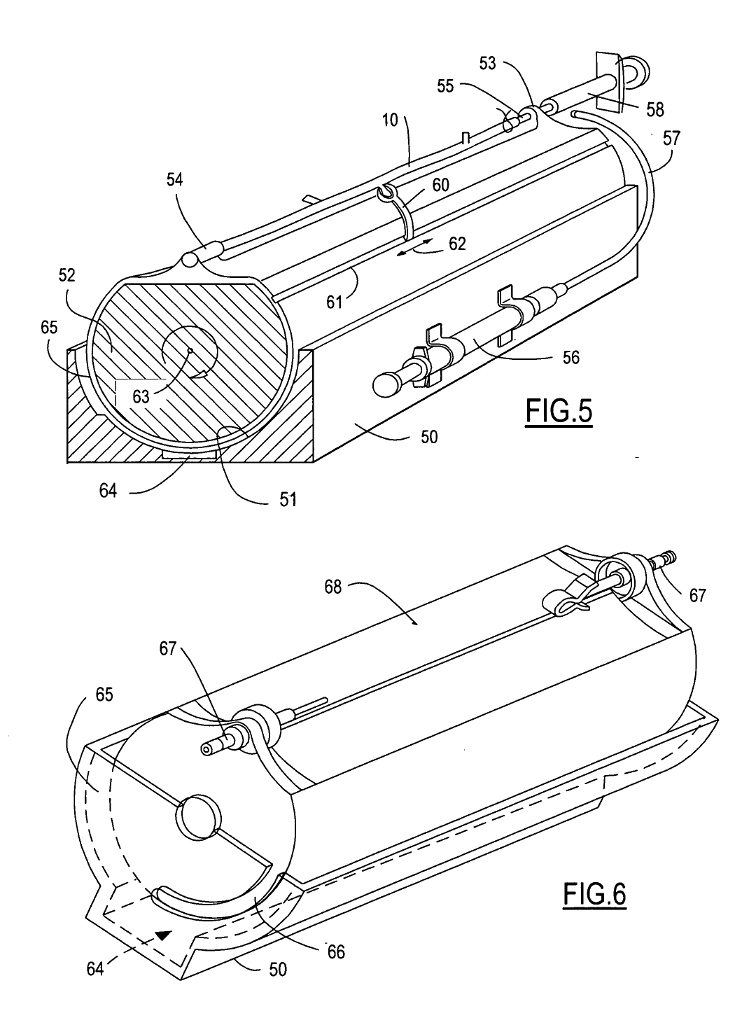

FIG. 5 shows an alternative embodiment wherein a reservoir is incorporated into a base 50. Thus, base 50 includes a recess 51 for receiving a rotatable member 52 carrying a first fixture 53 for receiving blood vessel 10 and a swivel clamp 54 at the opposite end of member 52 for supporting the other end of blood vessel 10. A fluid supply needle 55 in first fixture 15 receives fluid from a syringe 56 and feed line 57 as described in the previous embodiment, or from an optional syringe 58 connected coaxially with needle 55. A branch clamp 60 is able to clamp branch stubs at its proximal end and is slideably mounted in a longitudinal groove 61 of member 52 at its proximal end. Clamp 60 can be slid back and forth in the direction shown by arrow 62 to coincide with branch stubs to be ligated.

After processing of blood vessel 10, rotatable member 52 may be rotated around its axis 63 so that after 180 degrees of rotation, blood vessel 10 resides in a bath chamber 64 running longitudinally at the bottom of recess 51 in base 50. Recess 51 may include a cut out area 65 to accommodate the space needed by branch clip 60 when member 52 is rotated to place blood vessel 10 into bath 64.

FIG. 6 shows an embodiment similar to FIG. 5 for providing an integral bath chamber 64. Base 50 includes an end cover 65 having an arcuate slot 66 in the near end and a mirror image slot in the opposite end (not shown). When rotated, fixture components 67 (e.g., an extension of the mounting boss for connecting with a feed line at one end and a protrusion of a clamp shaft at the other end), traverse arcuate slots 66 allowing rotatable member 68 to flip over so that the blood vessel resides within bath chamber 64. A locking mechanism such as detents (not shown) in a hinge mechanism 69 provide for locking member 68 into a vessel processing position or a vessel immersing position.

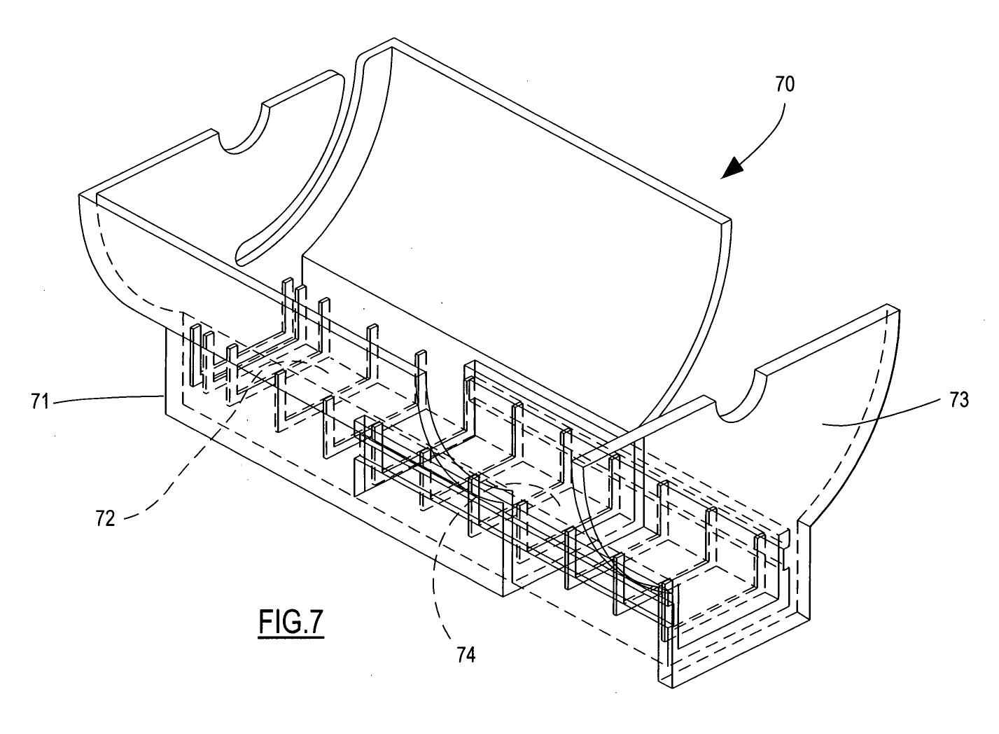

In the embodiment shown in FIG. 6, rotatable member 68 and base 50 have a fixed length. An adjustable distance between fixtures can be obtained by making the fluid supply needle and/or the clamp repositionable within their fixture. Member 68 and base 50 could also be constructed using telescopic parts in order to provide the adjustable distance. For example, FIG. 7 shows a telescopic base 70 with a first member 71 having a channel section 72. A second member 73 has a channel section 74 that is sealingly retained in channel section 72. Base 70 and a similarly telescoping rotatable member attached to members 71 and 73 would be extended or contracted together according to the length of a blood vessel being processed and the length of the immersion bath automatically accommodates the telescoped size of the rotatable member. Channel sections 72 and 74 would be filled with the appropriate volume of saline or other solution at the point of use.

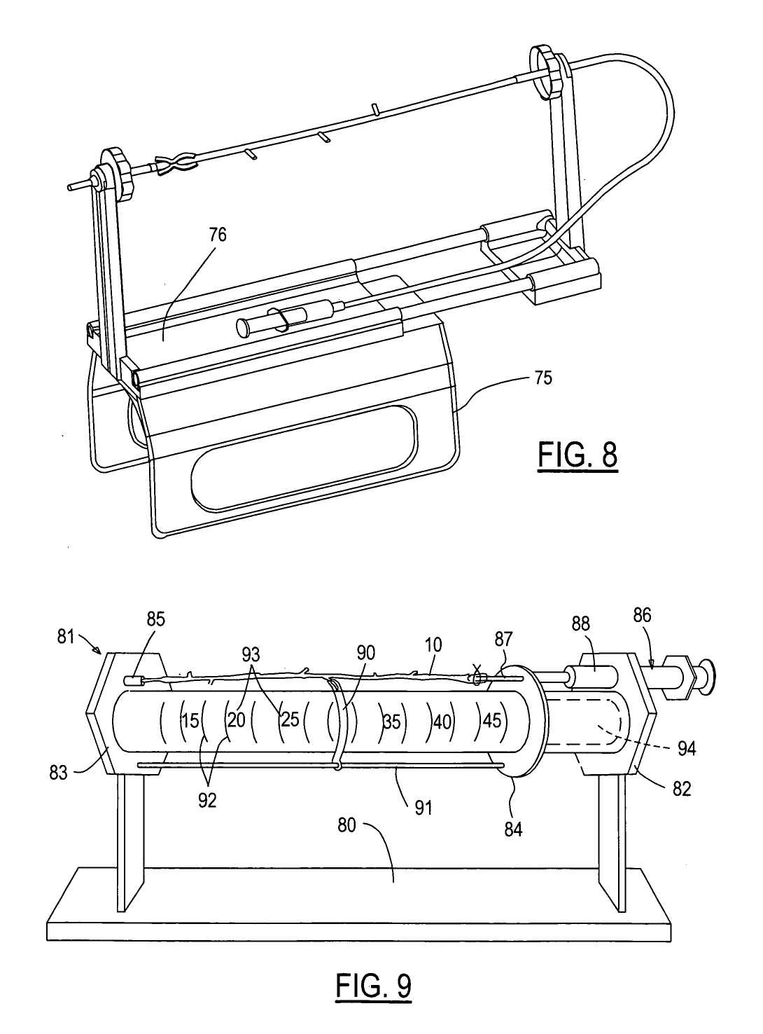

FIG. 8 shows a C-shaped sleeve 75 mounted underneath a base 76 according to any embodiment of a vessel holder device of the present invention. Sleeve 75 is placed over a limb of the patient (e.g., the opposite leg from the one used to harvest a saphenous vein) for convenient processing of the blood vessel near the place from which it is harvested and near the place where it will be reinserted into the patient.

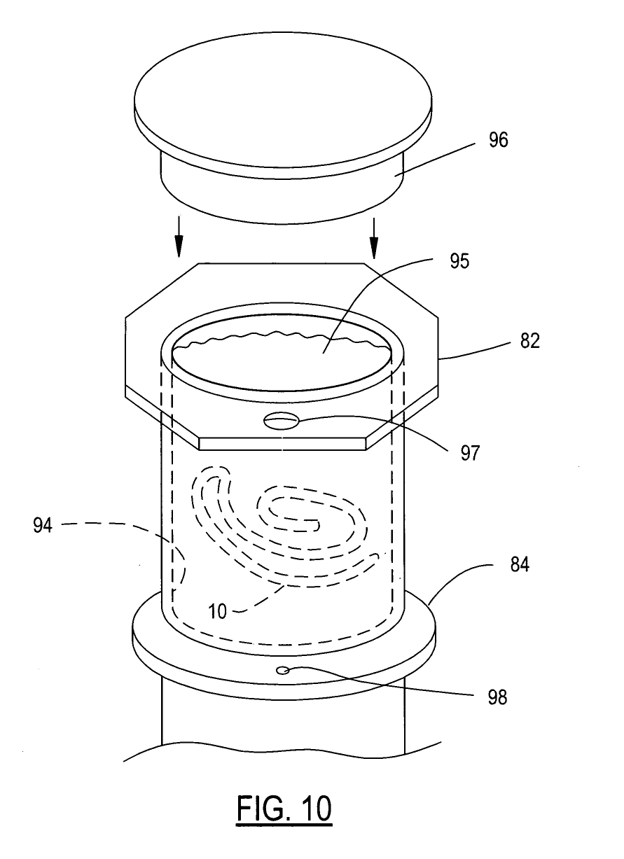

FIGS. 9 and 10 show yet another embodiment of a vessel holder. A base 80 supports a cylindrical tube member 81 having end flanges 82 and 83 and an intermediate flange 84. A clamp 85 is mounted to flange 83 for retaining one end of blood vessel 10. A syringe 86 extends through an aperture 97 in flange 82 and has a needle 87 extending through an aperture 98 in flange 84 to retain the other end of blood vessel 10. A snug but movable fit of syringe 86 in the apertures can provide an adjustable distance to achieve the desired tension in blood vessel 10. A branch clamp arm 90 is slidably mounted on a support rod 91 that extends between flanges 83 and 84.

Along the central tubular body of member 81, a plurality of graduations 92 are scribed or otherwise created at predetermined intervals. A plurality of labels 93 are also applied to member 81 to assist in determining the length of a vessel mounted to the vessel holder.

Tube member 81 includes a cavity 94 for providing a bath for storing blood vessel 10 after processing. As shown in FIG. 10, the vessel holder may be placed on end so that cavity 94 may be filled with a saline solution to a sufficient depth to immerse blood vessel 10. Cavity 94 preferably only penetrates into tube member 81 for a short distance (e.g., to the vicinity of flange 84) rather that extending the full length of member 81. A stopper 96 is provided for enclosing cavity 94 to further protect blood vessel 10 and/or to allow pre-filling with saline solution.

What is claimed is:

1. A vessel holder for supporting a blood vessel during processing for use in bypass graft surgery, comprising:
a base;
a first fixture mounted on the base for holding a first end of the blood vessel and having a fluid supply needle for inserting into the first end of the blood vessel; and
a second fixture mounted on the base a predetermined distance from the first fixture for holding a second end of the blood vessel, wherein the predetermined distance is adjustable for holding the blood vessel under tension.

2. The vessel holder of claim 1 further comprising:
a fluid conduit for coupling a source of pressurized solution to the fluid supply needle so that pressurized solution is injected into the blood vessel during processing.

3. The vessel holder of claim 2 further comprising:
a syringe for providing the pressurized solution as a saline solution.

4. The vessel holder of claim 2 further comprising:
a pressure gauge coupled to the fluid conduit for displaying a pressure indication.

5. The vessel holder of claim 1 wherein the base has an adjustable length for controlling the predetermined distance between the first and second fixtures.

6. The vessel holder of claim 1 wherein the first and second fixtures are rotatable along a longitudinal axis aligned with the blood vessel for accessing each side of the blood vessel during processing.

7. The vessel holder of claim 1 wherein the blood vessel includes branch stubs to be closed during processing and wherein the vessel holder further comprises:
a branch clamp mounted to the base and repositionable between the first and second fixtures for selectably retaining a branch stub.

8. The vessel holder of claim 1 further comprising a reservoir for temporarily bathing the blood vessel after processing.

9. The vessel holder of claim 8 wherein the reservoir is comprised of a recess in the base.

10. The vessel holder of claim 9 wherein the base comprises a rotatable section for rotating the blood vessel into the reservoir while still attached to the first and second fixtures.

11. The vessel holder of claim 1 wherein the base includes a semi-cylindrical frame adapted to fit over a limb of a patient from whom the blood vessel is harvested.

12. The vessel holder of claim 1 comprising graduation marks between the first and second fixtures for determining a length of the blood vessel.

13. A method of processing a blood vessel harvested from a patient for bypass graft surgery, the blood vessel having severed branch stubs along its length, the method comprising the steps of:
inserting one end of the blood vessel onto a fluid supply needle of a first fixture of a vessel holder, the vessel holder having a base supporting the first fixture;
affixing the other end of the blood vessel onto a second fixture of the vessel holder supported by the base;
adjusting the distance between the first and second fixtures to suspend the blood vessel under a tension;
sealing a first branch stub of the suspended blood vessel;
rotating the first and second fixtures around an axis substantially parallel to the suspended blood vessel to orient a further branch stub toward a user;
sealing the further branch stub; and
pressurizing the suspended blood vessel by injecting a solution into the suspended blood vessel through the fluid supply needle to check for leaks at the sealed branch stubs.

14. The method of claim 13 wherein the solution is injected from a syringe coupled to the fluid supply needle, wherein the vessel holder further includes a pressure gauge coupled to the fluid supply needle, and wherein the pressurizing step comprises:
manually operating the syringe by an amount that maintains a pressure detected by the pressure gauge below a predetermined pressure.

15. The method of claim 13 wherein the sealing steps each comprise:
clamping an end of a respective branch stub with a branch clamp supported by the base and axially repositionable between the first and second fixtures; and
tying off the respective branch stub with a suture.

16. The method of claim 13 further comprising the step of:
reconfiguring the vessel holder to bathe the suspended blood vessel in a solution contained in a reservoir until needed for the bypass graft surgery.

17. The method of claim 13 further comprising the steps of:
removing the blood vessel from the first and second fixtures after determining that no leaks are present through the sealed branch stubs; and
bathing the blood vessel in a solution contained in a reservoir within the base until needed for the bypass graft surgery.

18. The method of claim 13 further comprising the step of:
inspecting graduation marks on the vessel holder to determine a length of the blood vessel.

19. A vessel holder for supporting a blood vessel during processing for use in bypass graft surgery, comprising:
a base;
a first fixture mounted on the base for holding a first end of the blood vessel and having a fluid supply needle for inserting into the first end of the blood vessel; and
a second fixture mounted on the base a predetermined distance from the first fixture for holding a second end of the blood vessel;
wherein the first and second fixtures are rotatable around the longitudinal axis of the blood vessel to selectably orient branch stubs on the blood vessel at a desired rotational position.

20. The vessel holder of claim 19 wherein the predetermined distance is adjustable for holding the blood vessel under tension.

21. A vessel holder for supporting a blood vessel during processing for use in bypass graft surgery, comprising:
a base;
a first fixture mounted on the base for holding a first end of the blood vessel and having a fluid supply needle for inserting into the first end of the blood vessel;
a second fixture mounted on the base a predetermined distance from the first fixture for holding a second end of the blood vessel; and
a fluid conduit for coupling a source of pressurized solution to the fluid supply needle so that pressurized solution is injected into the blood vessel during processing.
